(19) United States
(12) Patent Application Publication
(10) Pub. No.: US 2009/0163889 A1
(43) Pub. Date: Jun. 25, 2009

(54) Biodelivery System for Microtransponder Array

(75) Inventors: Lawrence James Cauller, Plano, TX (US); Richard Weiner, Dallas, TX (US)

Correspondence Address: DAVID C. CAIN, PO Box 863387, Plano, TX, 75086-3387, US

(73) Assignee: Microtransponder, Inc., Dallas, TX (US)

(21) Appl. No.: 12/323,952

(22) Filed: Nov. 26, 2008

Related U.S. Application Data
(60) Provisional application No. 60/990,278, filed on Nov. 26, 2007.

(51) Int. Cl. A61M 5/32; A61M 5/315

(52) U.S. Cl. 604/506; 604/218

(57) ABSTRACT

Methods and devices for hypodermic implanting micro-transponders into a body. The method includes preassembling an array of micro-transponders into a cannula which is configured for tissue penetration and injection.

### CROSS-REFERENCE

Priority is claimed from U.S. provisional application 60/990,278 filed on Nov. 26, 2007, which is hereby incorporated by reference.

### BACKGROUND

The present application relates to biological implanting procedure and device, and more particularly to a bio-delivery system for micro devices, specifically to bio-delivery system for wireless micro-transponders.

### BRIEF DESCRIPTION OF THE DRAWINGS

The disclosed inventions will be described with reference to the accompanying drawings, which show important sample embodiments of the invention and which are incorporated in the specification hereof by reference, wherein:

FIG. 1 shows an expanded view of an example of a micro-transponder bio-delivery system.

FIG. 2 shows an example of loading a hypodermic cannula with micro-transponder array during manufacturing process.

FIG. 3 shows an example of a micro-transponder ejection system.

FIG. 4 shows a cross-sectional view of an example of micro-transponder implantation process.

FIG. 5 shows a cross-sectional view of a micro-transponder ejection system immediately after an implantation process.

FIG. 6 shows an example of a micro-transponder array.

FIG. 7(a) shows a side view of the micro-transponder array of FIG. 6.

FIG. 7(b) shows a plan view of the micro-transponder array of FIG. 6.

FIG. 8 shows another example of a micro-transponder array.

FIG. 9(a) shows a side view of the micro-transponder array of FIG. 8.

FIG. 9(b) shows a plan view of the micro-transponder array of FIG. 8.

FIG. 10 is a block diagram depicting a micro-transponder system, in accordance with an embodiment.

### DETAILED DESCRIPTION OF SAMPLE EMBODIMENTS

Note that the points discussed below may reflect the hindsight gained from the disclosed inventions, and are not necessarily admitted to be prior art.

Implanting devices, such as electrodes, into the body such as the brain, or the muscle without major surgery has remained a challenge. In addition, the incorporation of foreign matter and/or objects into the human body presents various physiological complications. For example, the size and extension of the implanted devices and wires extending therefrom may substantially restrict the ways available to implant such devices.

The invention of micro devices makes it very promising in non-invasive implanting because of their extreme small sizes. However, special concerns may be taken as to orientation control and quantitative control. Concerns over removing the implanted devices are also valid.

For electronic implants one of the primary modes of failure is the foreign body response (FBR). Generally this response is triggered by adsorbance and denaturation of proteins on the implanted substrate, followed by activation of neutrophils and macrophages. Macrophages that are unable to phagocytose the implant begin fusing to form foreign body giant cells, which release free radicals that may damage the implanted device. Often this is followed by the formation of a fibrous or glial scar which encapsulates the device and segregates it from the target tissue.

There is great need in providing an efficient bio-delivery system for micro electronic devices.

The present application discloses new approaches to deliver micro-transponders or micro-stimulators in an easy to operate manner into a biological system, and that is quantitatively controllable.

A micro-transponder is a wireless ‘micro-transponders’ that combines interface micro-electronics with the basic RFID design. The wireless performance of any such transponder is a function of two basic electronic components, its inductor coil, LT, and its resonance capacitor, CT, whose values are primarily determined by their size. The neural interface may include neural stimulators, such as an electrode. The ranging sizes of micro-transponders are from a few hundred square μm to around one square mm. The micro-transponder is capable of wireless power induction and effective coupling at a distance. Examples of micro-transponders are described in detail in the U.S. provisional application 60/990,278 filed on Nov. 26, 2007, which has been incorporated by reference in entirety. Micro-transponder devices may be arranged in a spatially defined pattern as an array. An array of micro-transponder may comprise plurality of micro-transponders arranged, for example, in parallel as a strip, a plaque, or in any other patterns or shapes, or numbers or forms. Micro-transponders in an array may or may not be physically linked.

However, it is contemplated and intended that the techniques in this disclosure may be used and applied to other suitable micro devices for in vivo delivery as it may be obvious for a person skilled in the art.

In one embodiment, individual micro-transponders are linked together by a durable non-fouling material to form a core strip.

In one embodiment, linked micro-transponder arrays are embedded within a biocompatible matrix. The core material is fabricated from a material (or coated with) that will minimize adhesion with the matrix and in-growing tissue.

In one embodiment, the micro-transponder arrays are loaded into an injection system during the manufacturing process. The injection system comprises a cannula, stylet, handle, and spring. The cannula will act as an introducer needle, protecting the array during insertion into the tissue.

The disclosed innovations, in various embodiments, provide one or more of at least the following advantages. However, not all of these advantages result from every one of the innovations disclosed, and this list of advantages does not limit the various claimed inventions.

Easy to operate and control;
Minimal invasiveness and complication;
High efficiency and high biocompatibility.

The numerous innovative teachings of the present application will be described with particular reference to presently preferred embodiments (by way of example, and not of limitation). The present application describes several inventions, and none of the statements below should be taken as limiting the claims generally.

For simplicity and clarity of illustration, the drawing figures illustrate the general manner of construction, and description and details of well-known features and techniques may be omitted to avoid unnecessarily obscuring the invention. Additionally, elements in the drawing figures are not necessarily drawn to scale, some areas or elements may be expanded to help improve understanding of embodiments of the invention.

The terms “first,” “second,” “third,” “fourth,” and the like in the description and the claims, if any, may be used for distinguishing between similar elements and not necessarily for describing a particular sequential or chronological order. It is to be understood that the terms so used are interchangeable. Furthermore, the terms “comprise,” “include,” “have,” and any variations thereof, are intended to cover non-exclusive inclusions, such that a process, method, article, apparatus, or composition that comprises a list of elements is not necessarily limited to those elements, but may include other elements not expressly listed or inherent to such process, method, article, apparatus, or composition.

For micro electronic devices to be effective inside a biological body, many times it requires multiple numbers of devices to work on different body spots independently or together synergistically. Micro-transponder is a micro-device when implanted into tissue, has at least one electrical connection to the tissue, and also has a wireless external interface. Because of their small sizes quantitatively implanting micro-devices in a specific body spot can be achieved by controlling the concentration of micro devices and by injection, and alternatively by surgical incisions.

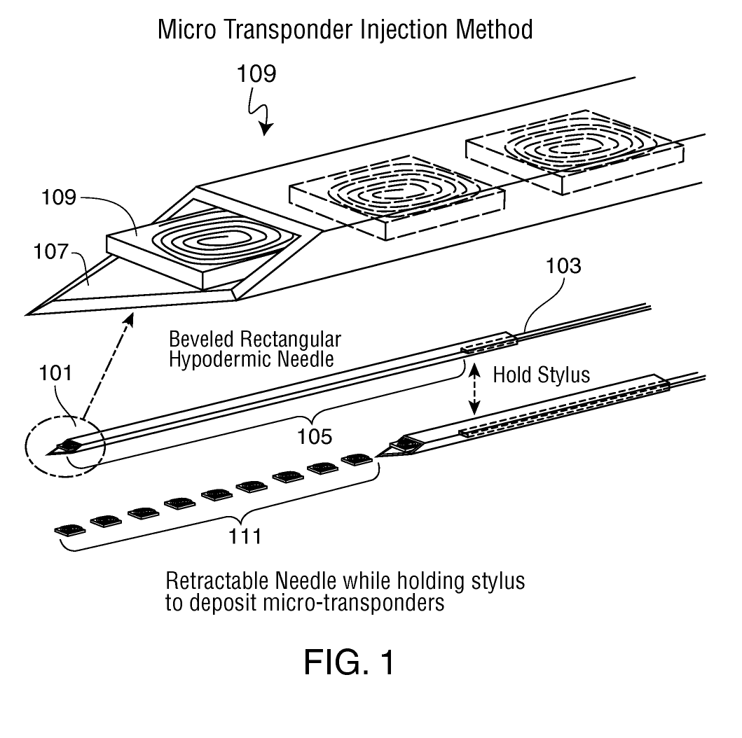

FIG. 1 shows an example of injection system 100 comprising a loaded cannula 105, stylet 103 that can push through the cannula. To safely insert a micro-stimulator/micro-transponder to a body location cannula 105 is designed to be square and small diameter as the introducer with tapered dilator that does not have sharp edges. The placement of micro-transponders or array of micro-transponders will likely be a drop-down placement. Cannula 105 can be open mouthed, and the front tip 101 may include an extruded edge 107 that can guide micro-transponders 109 into a target body location. Micro-transponders are deposited (111) by holding stylus 103 in place or retracting it more slowly while retracting the needle/cannula 105.

It is also contemplated and intended that cannula 105 may also have the ability to retrieve a micro device array immediately or during the next 8-10 days, without a cut-down or reinserting another cannula into the tissue.

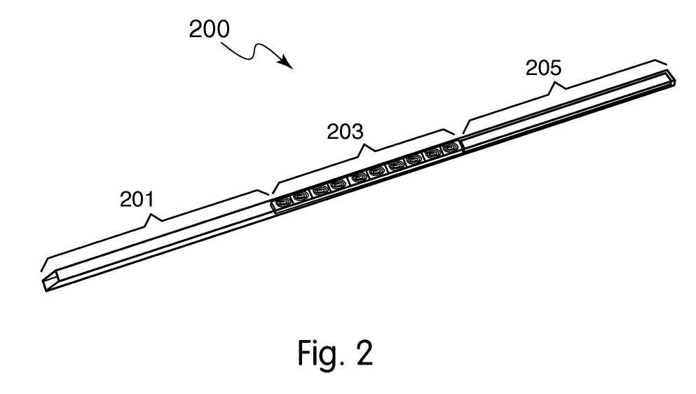

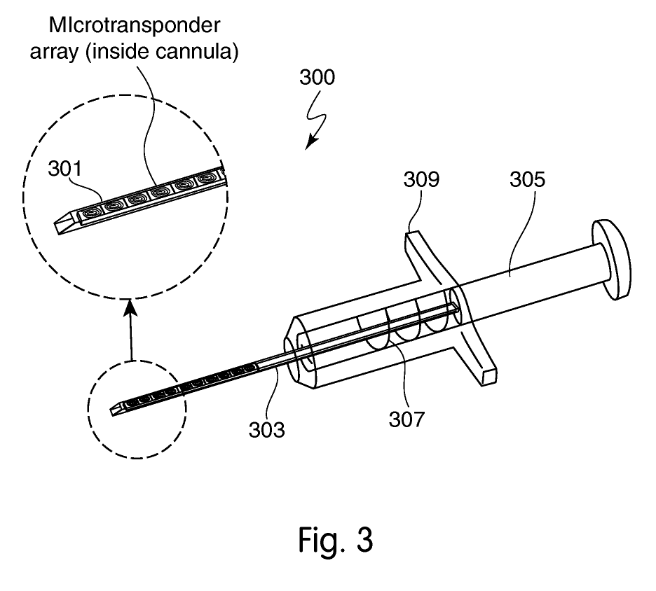

The array of micro-transponders is loaded into the injection system during the manufacturing process. FIG. 2 shows an example of pre-loading micro-transponder array 203 into cannula 201 with or without the attachment of stylet 205. Microtransponders can be packaged or suspended in a biocompatible medium, such as MatriGel™ or agarose, or PEG, or glycerol, or sodium water etc., to help pre-loading process. Microtransponders can also be packed into the cannula mechanically by hand or by machine, without any other medium. FIG. 3 shows another example of a pre-packaged injection system which has a stylet 303 attached to a syringe-like device which contains a handle holder 309, a spring 307 and a handle 305 for injection. The whole package is sterilized. The preloaded delivery system may be disposable and used only once. After the manufacturing process is completed, the array 301 will be ready for implantation after removal from the packaging.

The internal compression spring 307 will keep the injection system from accidentally dispensing the array during shipment and handling. A needle cap may be used to prevent accidental dispensing and sharps protection.

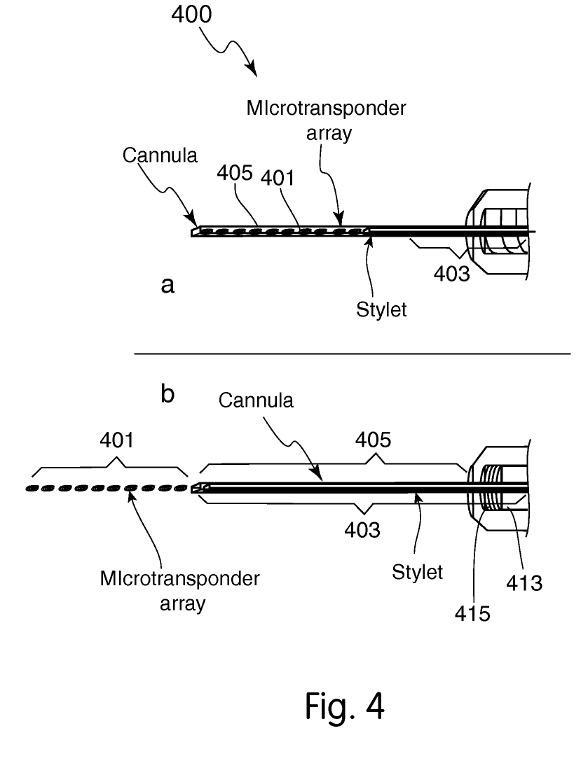

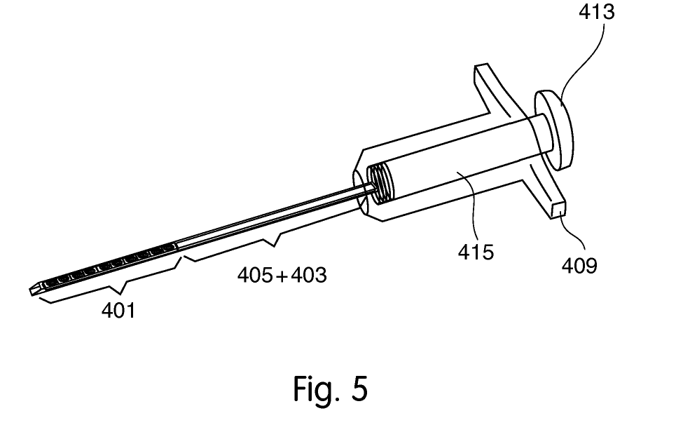

FIG. 4(a) shows a preloaded injection system with a relaxed spring. FIG. 4(b) shows that after inserting the needle/cannula 405 into the tissue, handle 413 is pushed, compressing the spring 415 and stylet 403 and thereby pushing micro-transponder array 401 into the tissue. After the injection into the tissue, handle holder 409 (FIG. 5) may be used to retract cannula 405, leaving the injected array in the tissue. FIG. 5 shows an example look of the injection system immediately after the micro-transponder ejection.

Materials for the construction of the injection system are biocompatible, for example the cannula and stylet can be stainless steel and the handle and the handle holder can be acrylonitrile butadiene styrene (ABS), polycarbonate, or polyurethane. The stylet may also be made of bio-compatible plastics. Sterilization can be conducted and verified according to standard GMP procedure required by the FDA for the intended production environment and processes and purposes.

During the pre-loading process, the cannula and stylet may need to be fabricated from custom extruded material, so that there is limited space between the array and the walls of the cannula. A biocompatible lubrication material, such as polyethylene glycol (PEG), may be used to reduce the friction between the array and the cannula.

The foreign body response (FBR) is one of the primary modes of failure for electrical implants. Generally this response is triggered by adsorbance and denaturation of proteins on the implanted substrate, followed by activation of neutrophils and macrophages. Macrophages that are unable to phagocytose the implant begin fusing to form foreign body giant cells, which release free radicals that may damage the implanted device. Often this is followed by the formation of a fibrous or glial scar which encapsulates the device and segregates it from the target tissue.

It has been shown that both porous scaffold materials and non-fouling coating can reduce the host FBR. A multitude of unique materials and designs have been tested for this purpose. It is desirable to not only reduce the FBR, but also to encourage intimate contact between the implanted devices and target tissues. The primary drawback with previous strategies encouraging tissue integration with implants, is that they can only be removed by excision of actual tissue. This application discloses a novel design to both encourage tissue integration and facilitate removal of devices in the event of failure, patient paranoia, or completion of therapy.

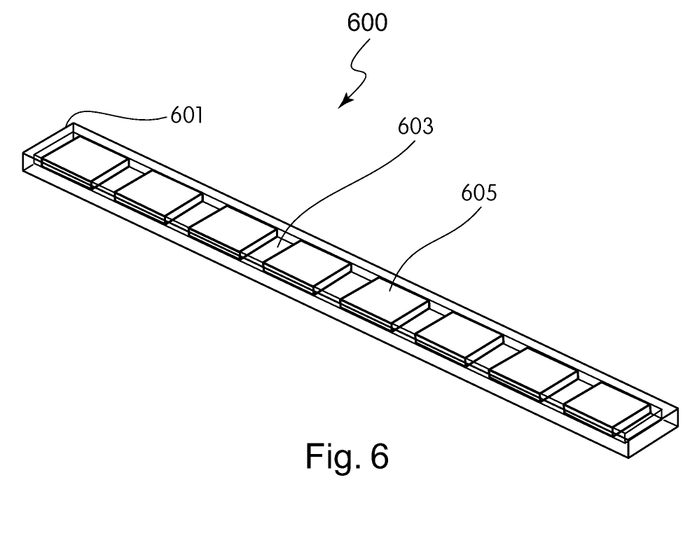

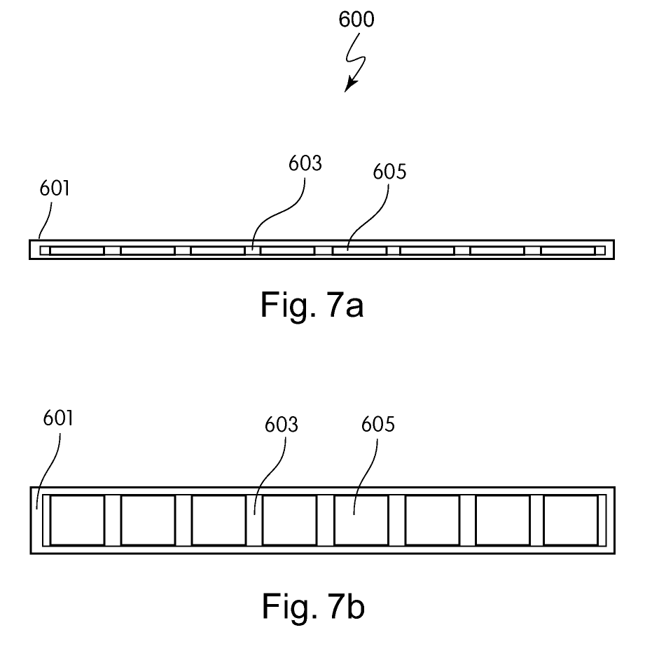

To accomplish this end, as shown in FIGS. 6 and 7, a plurality of individual micro-transponders 605 can be linked together to form an array and a core strip 603 by a durable non-fouling material, for example, SU8 with the surface coated with a lubricious, protein adsorption preventing, “stealth” material. The core strip is then embedded within a porous scaffold 601. The core material will be fabricated from a material (or coated with) that will minimize adhesion with the scaffold and in-growing tissue. Biocompatible material that will encourage growth of surrounding tissue up to the implanted devices and exposed SU8 is used for the scaffold which is designed in a manner to both minimize FBR and encourage the penetration of endothelial cells and neurites. By separating the tissue integrating scaffolding from the solid core, removal of the actual devices can be carried out simply by making an incision to expose the end of the core, grasping it, and then sliding it out from the scaffolding.

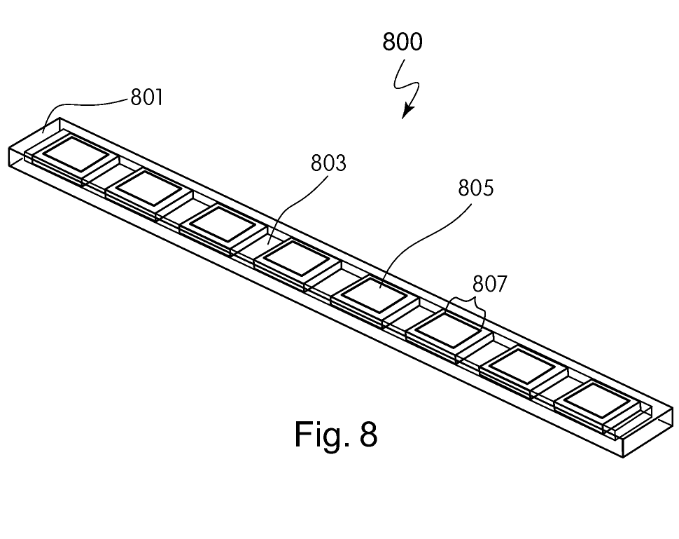

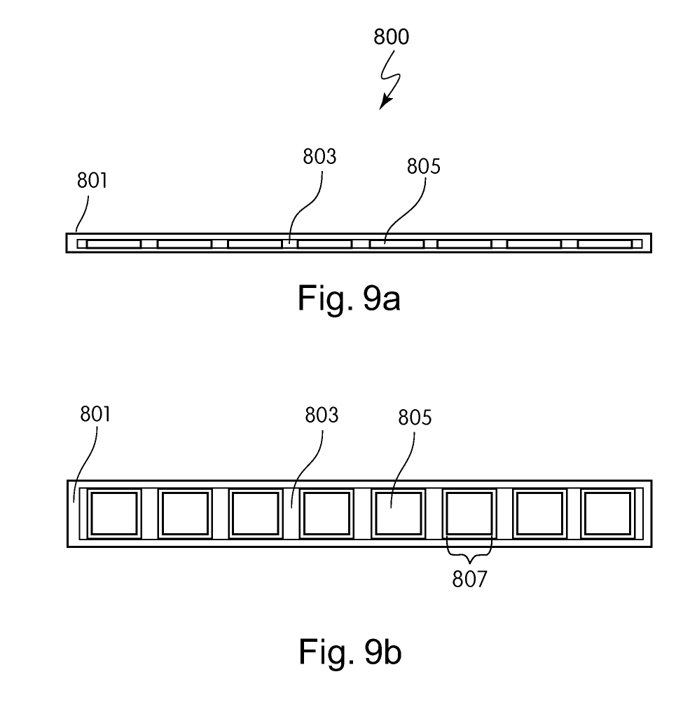

Another embodiment of the micro-transponder array is shown in FIGS. 8 and 9. The core strip 803 is a strong strip containing an embedded array of individual micro-transponders, where the superior and inferior electrodes of micro-transponders are exposed through “windows” 807. Electrode surfaces and strip may be coated with a lubricious, protein adsorption preventing, “stealth” material. The core strip is then embedded within a porous scaffold/matrix 801 that the scaffolding will extend into the “windows.” Other durable and more flexible material than SU8 can be used, and embedded micro-transponders can be better protected. Electrodes of micro-transponders 805 can be totally isolated from proteins/tissues, but still affect ions in solution.

Micro-transponders may be physically unlinked while inside the cannula and stored in low temperature, such as around 4° C.; the physically linked array may be formed after the injection by a biocompatible get like material, such as Matrigel™ (a product of BD Biosciences, Inc), that solidifies when exposed to higher temperature, such as body temperature, and the space between each micro-transponder may be adjusted by the pushing speed.

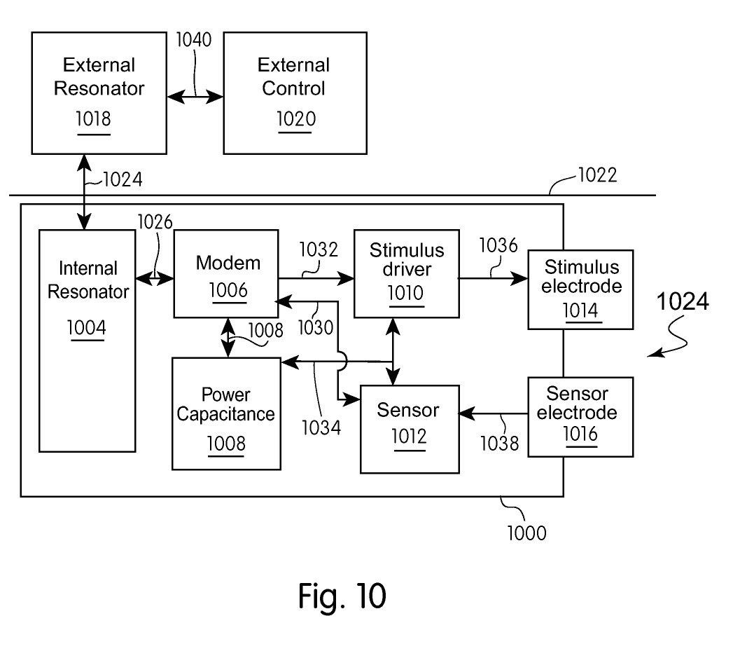

Other designs suited to applications such as vagus nerve stimulation (which may be applied to peripheral nerves in general) may also be adopted and accommodated. A design shown in FIG. 10 that consists of a flexible helix containing exposed micro-transponders on the inner surface, arranged in a manner such that all coils lay parallel to the overlying skin. The array of micro-transponders may have linked electrodes so that they function as a single stimulator, to maximize stimulation around the entire periphery of the nerve. Sizes of micro-transponders can be formed square form-factors of sizes (microns) such as 500×500; 1000×1000; 2000×2000, in rectangular form-factors of sizes (microns) such as 200×500; 250×750; 250×1000, circular and or other shapes.

With reference to FIG. 10, a block diagram depicts a microtransponder 1000 in accordance with an embodiment. The microtransponder 1000 may be implanted in tissue 1024 beneath a layer of skin 1022. The microtransponder 100 may be used to sense neural activity in the tissue 1024 and communicate data to an external control 1020 in response. The microtransponder 1000 may be used to provide electrical stimulation to the tissue 1024 in response to a signal from an external control 1020. The electrodes 1014 and 1016 may be designed to enhance the electrical interface between the electrodes 1014 and 1016 and neurons of peripheral nerves.

The microtransponder 1000 may wirelessly interact with other systems. The microtransponder 1000 may interact via direct electrical connection with other systems. Typically, the microtransponder 1000 interacts wirelessly with an external control system 1020 including an external resonator 1018. The microtransponder 1000 may communicate via a direct electrical connection with other microtransponders (not shown) implanted within the body.

The microtransponder 1000 enables delivery of electrical signals to peripheral nerves. These signals may be configured to stimulate peripheral nerves distributed throughout subcutaneous tissue 1024. The microtransponder 1000 enables the detection of electrical signals in peripheral nerves. The detected electrical signals may be indicative of neural spike signals.

Microtransponder 1000 includes an internal resonator 1004. The internal resonator 1004 might be connected to a modulator-demodulator 1006, to modulate information onto outgoing signals and/or retrieve information from incoming signals. The modulator-demodulator 1006 may modulate or demodulate identification signals. The modulator-demodulator 1006 may demodulate trigger signals. The modulator-demodulator 1006 may receive signals from an impulse sensor 1012. The modulator-demodulator 1006 may provide trigger signals or other data to a stimulus driver 1010. The impulse sensor 1012 may be connected to a sensor electrode 1016. The impulse sensor 1012 may generate a signal when a current is detected at the sensor electrode 1016. The stimulus driver 1010 may be connected to stimulus electrodes 1014. The stimulus driver 1010 typically generates a stimulation voltage between the stimulus electrodes 1014 when a trigger signal is received.

The internal resonator 1004 provides energy to a power storage capacitance 1008, which stores power received by the internal resonator 1004. The power capacitance 1008 may provide power 1034 to the other components, including the stimulus driver 1010, the impulse sensor 1012 and the modem 1006.

In operation, an external control 1020, typically a computer or other programmed signal source, may provide commands 1040 regarding sensing or stimulation for the microtransponder 1000. The commands 1040 are provided to an external resonator 1018 and may initiate stimulation cycles, poll the devices, or otherwise interact with the microtransponder 1000. The external resonator 1018 is tuned to resonate at the same frequency, or a related frequency, as the internal resonator 1004. Signal 1026 are generated by the external resonator 1018, resonated at the tuned frequency. The signal 1026 may be a power signal without any modulated data. The signal 1026 may be a power signal including modulated data, where the modulated data typically reflects commands 1040 provided by the external control 1020 such as identification information or addresses. It should be recognized that a power signal without modulated data may communicate timing data, such as a trigger signal, in the presentation or timing of the power signal.

The internal resonator 1004 receives signals 1026 from the external resonator 1018. The internal resonator 1004 provides a received signal 1026 to the modulator-demodulator (modem) 1006. The modem 1006 may demodulate instructions 1032 from the received signal. Demodulated instructions 1032 may be provided to the stimulus driver 1010. The modem 1006 may pass the power signal 1028 to the power capacitance 1008. The power capacitance 1008 may store the power signal 1028. The power capacitance 1008 may provide power to the stimulus driver 1010. The power capacitance 1008 may provide power to the impulse sensor 1012. The stimulus driver 1010 may provide a stimulus signal 1036 to the stimulus electrode 1014. The stimulus driver 1010 may provide a stimulus signal 1036 to the stimulus electrode 1014 in response to an instruction 1032. The stimulus driver 1010 may provide a stimulus signal 1036 to the stimulus electrode 1014 in response to a power signal 1034.

The modem 1006 may provide an instruction 1030 to impulse sensor 1012. When an impulse is sensed in the tissue 1024, the sensor electrode sends an impulse signal 1038 to impulse sensor 1012. The impulse sensor 1012 sends a sensed impulse signal 1030 to the modem 1006. In response to the sensed impulse signal 1012, the modem 1016 may modulate an identification signal 1026 onto a power signal 1028. The internal resonator 1004 generates a communication signal 1024 including a modulated identification signal 1026. The external resonator 1018 receives the communication signal 1024. Data 1040 is provided to the external control 1020.

According to various embodiments, there is provided a method for implanting a plurality of micro-transponders into a cellular matter, comprising the actions of pre-assembling a plurality of micro-transponders in a cannula to form a loaded-cannula wherein said cannula is configured to hold micro-transponder in a fixed orientation; and ejecting said microtransponders from said cannula into the cellular matter, to thereby form extended array of microtransponders.

According to various embodiments, there is provided a method for implanting micro-transponders into a cellular matter, comprising the actions of forming a plurality of micro-transponders to be a spatially arranged array; embedding said array in a bio-compatible packaging material to form a strip; loading said strip into a cannula; and ejecting said strip into a cellular matter.

According to various embodiments, there is provided a device for hypodermic micro-transponder delivery, comprising: a) a cannula pre-loaded with a micro-transponder array of plurality of microtransponders; and b) an ejection mechanism which is configured to push through said cannula; wherein said cannula is configured for tissue penetration and injection.

According to various embodiments, there is provided a kit for hypodermic micro-transponder delivery, comprising: a) a cannula; b) a micro-transponder array that is loadable to said cannula; and b) an ejection mechanism which is configured to push through said cannula; wherein said cannula is configured for injection.

According to various embodiments, there is provided: 36. A method for implanting micro-transponder into a cellular matter, comprising the actions of pre-assembling a micro-transponders in a cannula to form a loaded-cannula wherein said micro-transponder has no local power source and said micro-transponder has a size of less than 1 square mm; and ejecting said microtransponders from said cannula into the cellular matter.

According to various embodiments, there is provided methods and devices for hypodermic implanting micro-transponders into a body. The method includes preassembling an array of micro-transponders into a cannula which is configured for tissue penetration and injection.

### MODIFICATIONS AND VARIATIONS

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

The shape of the cannula, its width, thickness and length can vary for different purposes and clinic uses. For example, for deep tissue injection, the cannula may be made of strong material of sharper edge with a long extended body. And the microtransponders may be linked physically to form an array.

The following applications may contain additional information and alternative modifications: Attorney Docket No. MTSP-29P, Ser. No. 61/088,099 filed Aug. 12, 2008 and entitled “In Vivo Tests of Switched-Capacitor Neural Stimulation for Use in Minimally-Invasive Wireless Implants; Attorney Docket No. MTSP-30P, Ser. No. 61/088,774 filed Aug. 15, 2008 and entitled “Micro-Coils to Remotely Power Minimally Invasive Microtransponders in Deep Subcutaneous Applications”; Attorney Docket No. MTSP-31P, Ser. No. 61/079,905 filed Jul. 8, 2008 and entitled “Microtransponders with Identified Reply for Subcutaneous Applications”; Attorney Docket No. MTSP-33P, Ser. No. 61/089,179 filed Aug. 15, 2008 and entitled “Addressable Micro-Transponders for Subcutaneous Applications”; Attorney Docket No. MTSP-36P Ser. No. 61/079,004 filed Jul. 8, 2008 and entitled “Microtransponder Array with Biocompatible Scaffold”; Attorney Docket No. MTSP-38P Ser. No. 61/083,290 filed Jul. 24, 2008 and entitled “Minimally Invasive Microtransponders for Subcutaneous Applications” Attorney Docket No. MTSP-39P Ser. No. 61/086,116 filed Aug. 4, 2008 and entitled “Tintinnitus Treatment Methods and Apparatus”; Attorney Docket No. MTSP-40P, Ser. No. 61/086,309 filed Aug. 5, 2008 and entitled “Wireless Neurostimulators for Refractory Chronic Pain”; Attorney Docket No. MTSP-41P, Ser. No. 61/086,314 filed Aug. 5, 2008 and entitled “Use of Wireless Microstimulators for Orofacial Pain”; Attorney Docket No. MTSP-42P, Ser. No. 61/090,408 filed Aug. 20, 2008 and entitled “Update: In Vivo Tests of Switched-Capacitor Neural Stimulation for Use in Minimally-Invasive Wireless Implants”; Attorney Docket No. MTSP-43P, Ser. No. 61/091,908 filed Aug. 26, 2008 and entitled “Update: Minimally Invasive Microtransponders for Subcutaneous Applications”; Attorney Docket No. MTSP-44P, Ser. No. 61/094,086 filed Sep. 4, 2008 and entitled “Microtransponder MicroStim System and Method”; Attorney Docket No. MTSP-30, Ser. No. ______, filed ______ and entitled “Transfer Coil Architecture”; Attorney Docket No. MTSP-31, Ser. No. ______, filed ______ and entitled “Implantable Driver with Charge Balancing”; Attorney Docket No. MTSP-28, Ser. No. ______, filed ______ and entitled “Implantable Transponder Systems and Methods”; Attorney Docket No. MTSP-46, Ser. No. ______, filed ______ and entitled “Implanted Driver with Resistive Charge Balancing”; Attorney Docket No. MTSP-47, Ser. No. ______, filed ______ and entitled “Array of Joined Microtransponders for Implantation”; and Attorney Docket No. MTSP-48, Ser. No. ______, filed ______ and entitled “Implantable Transponder Pulse Stimulation Systems and Methods” and all of which are incorporated by reference herein.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

1. A method for implanting a plurality of micro-transponders into a cellular matter, comprising the actions of:
pre-assembling a plurality of micro-transponders in a cannula to form a loaded-cannula wherein said cannula is configured to hold micro-transponder in a fixed orientation; and
ejecting said microtransponders from said cannula into the cellular matter, to thereby form extended array of microtransponders.

2. The method of claim 1, wherein said micro-transponders are smaller than 1 square millimeter.

3. The method of claim 1, wherein said loaded-cannula is sterilized and disposable after first use.

4. The method of claim 1, wherein said microtransponders are packaged in a biocompatible material made of Matrigel™.

5. The method of claim 1, wherein the step of pre-assembling is performed during the manufacturing process.

6. The method of claim 1, wherein said micro-transponders form a spatially arranged array with other micro-transponders in said cannula during the step of pre-assembling.

7. The method of claim 1, wherein said micro-transponders forms a spatially arranged array with other micro-transponders in the cellular tissue after the step of ejecting.

8. The method of claim 1, wherein said micro-transponders are wirelessly and electro-magnetically connected to an external device.

9. The method of claim 1, wherein said cannula has a flat extrusion at the free end that guides said micro-transponders disposition in the cellular matter.

10.-12. (canceled)

13. A method for implanting micro-transponders into a cellular matter, comprising the actions of:
forming a plurality of micro-transponders to be a spatially arranged array;
embedding said array in a bio-compatible packaging material to form a strip;
loading said strip into a cannula; and
ejecting said strip into a cellular matter.

14. The method of claim 13, wherein said individual micro-transponders are smaller than 1 square millimeter.

15. The method of claim 13, wherein said loaded-cannula are sterilized and disposable after first use.

16. The method of claim 13, wherein said biocompatible material is made of SU8.

17. The method of claim 13, wherein the step of loading is performed during the manufacturing process.

18. The method of claim 13, wherein said micro-transponders are suspended in Matrigel™ before the step of forming an array.

19. The method of claim 13, wherein said micro-transponders are wirelessly and electro-magnetically connected to a device.

20.-22. (canceled)

24. A device for hypodermic micro-transponder delivery, comprising:
a) a cannula pre-loaded with a micro-transponder array of plurality of microtransponders; and
b) an ejection mechanism which is configured to push through said cannula; wherein said cannula is configured for tissue penetration and injection.

25. The device of claim 24, wherein said cannula is configured to accommodate with the shape of said micro-transponder array.

26. The device of claim 24, wherein said cannula is configured such that its free end opening is shaped to guide the orientation of said microtransponder array during a hypodermic delivery.

27. The device of claim 24, wherein at least one said micro-transponder has a width less than 1 mm.

28.-36. (canceled)
